(19) United States
(12) Patent Application Publication
(10) Pub. No.: US 2024/0229007 A1
(43) Pub. Date: Jul. 11, 2024

(54) INACTIVATED VACCINES & NUCLEIC ACID APTAMER THERAPEUTICS DERIVED THEREFROM

(71) Applicant: Universal Stabilization Technologies, Inc., San Diego, CA (US)

(72) Inventor: Victor Bronshtein, San Diego, CA (US)

(73) Assignee: Universal Stabilization Technologies, Inc., San Diego, CA (US)

(21) Appl. No.: 17/313,798

(22) Filed: May 6, 2021

Related U.S. Application Data
(60) Provisional application No. 63/021,044, filed on May 6, 2020.

(51) Int. Cl. C12N 13/00; A61K 39/00; A61K 39/02; A61K 39/12

(52) U.S. Cl. CPC C12N 13/00; A61K 39/0002; A61K 39/02; A61K 39/107; A61K 39/12; A61K 2039/521; A61K 2039/53; A61K 2039/6087

(57) ABSTRACT

Methods for producing dry thermostable inactivated vaccines with improved inactivation/killing without compromising antigenicity are disclosed. The methods provide for microbes to be irradiated with an ionizing radiation dose above 24 kGy without compromising the integrity of useful epitopes. To achieve enhanced inactivation, microorganisms (virions, bacterium, fungi, etc.) are first immobilized in a dry glassy matrix, then subsequently irradiated at elevated radiation dose while immobilized in the glass. Aptamers can be isolated from the inactivated microbes, such as inactivated SARS-COV-2, and modified or developed as therapeutics for neutralizing infectious disease. Additionally, non-neutralizing aptamers can be combined to produce an enhanced vaccine.

### CROSS-REFERENCE TO RELATED APPLICATIONS

This application claims benefit of priority with U.S. provisional application Ser. No. 63/021,044, filed May 6, 2020; the entire contents of which are hereby incorporated by reference for all purposes.

### STATEMENT REGARDING FEDERALLY SPONSORED RESEARCH OR DEVELOPMENT

None.

### BACKGROUND OF THE INVENTION

### Field of the Invention

The claimed invention relates to vaccines and therapeutics for use against infectious microbes; and more particularly, to such vaccines with improved inactivation without loss of antigenicity and nucleic acid aptamer therapeutics derived therefrom.

### Description of the Related Art

Globalization of the world economy and the modern itinerant way of life increases probability of spreading infectious disease outbreaks worldwide. The outbreak of SARS-COV-2 was first reported in Wuhan, China, in December 2019. The disease known as COVID-19 is caused by the SARS-COV-2 virus. Since the initial outbreak, the virus has rapidly spread throughout the world, and has infected and killed millions of individuals globally. The SARS-COV-2 outbreak presents unprecedented challenges related to global health issues, social and economic disruption, large numbers of deaths, and other disastrous consequences. There is an urgent need for quick development of vaccines as well as therapeutics to help treat patients and prevent further spread. There is also a need for vaccine enhancement strategies and formulation technologies that would allow reduced number of immunizations, increased ease of administration (i.e., self-administration), increased product stability to minimize cold chain requirements, and enhanced cost-effectiveness of vaccine manufacturing. This disclosure is directed to platform methodologies for rapid development of vaccines and therapeutics against, inter alia, quickly emerging infectious disease threats.

### SUMMARY OF THE INVENTION

In one aspect, a method is provided for producing a highly antigenic dry thermostable inactivated vaccine.

In another aspect, a method is provided for preparing a nucleic acid aptamer therapeutic composition for neutralizing infectious microbes.

Various embodiments of these aspects are disclosed and claimed herein.

### BRIEF DESCRIPTION OF THE DRAWINGS

FIG. 1 shows stability of PBV MVA vaccine at 37° C. compared to GMP FD MVA vaccine.

FIG. 2 shows stability of PBV YF vaccine at 37° C. compared to GMP FD YF vaccine.

FIG. 3 shows the preservation by vaporization process compared to conventional freeze drying.

FIG. 4 shows a method according to an embodiment of the invention.

### DETAILED DESCRIPTION

For purposes of explanation and not limitation, details and descriptions of certain preferred embodiments are hereinafter provided such that one having ordinary skill in the art may be enabled to make and use the invention in its various aspects and embodiments. These details and descriptions are representative only of certain preferred embodiments, however, and a myriad of other embodiments, which will not be expressly described, may be readily understood by one having skill in the art upon a thorough review of the instant disclosure. Accordingly, any reviewer of the instant disclosure should interpret the scope of the invention by the claims, as such scope is not intended to be limited by the embodiments described and illustrated herein.

For purposes herein, the terms “microorganism” and “microbe” are intended to be interchangeable, and to broadly include viruses (virions), bacteria (bacterium), vibrio, fungi and/or yeast. To this end, the microorganism selected for use in the described methods can be any known microorganism for which vaccine, therapeutic or other use is sought, as the methods apply to all microorganisms. However, given the current pandemic, the disclosure is tailored toward viruses without intent to limit the scope of the claimed invention.

Now, in accordance with various embodiments, a method of producing a dry thermostable inactivated vaccine is disclosed, the method comprising: (i) combining a microorganism preparation and a preservation formulation to form a vaccine suspension, the microorganism preparation comprising live virions or cellular microorganisms, the preservation formulation comprising amino acids, one or more non-reducing disaccharides, and one or more monosaccharide derivatives and/or sugar alcohols; (ii) drying the vaccine suspension to form a mechanically-stable glassy foam, said mechanically-stable glassy foam comprising: less than five percent residual water content, and a glass transition temperature greater than a maximum storage temperature, wherein the live virions or cellular microorganisms are immobilized in the mechanically-stable glassy foam; and (iii) exposing the mechanically-stable glassy foam to an ionizing radiation dose between 24 kGy and 48 kGy to produce an inactivated or killed vaccine.

The method can be enhanced where the preservation formulation comprises one part by weight monosaccharide derivatives and/or sugar alcohols and at least two parts by weight non-reducing disaccharides.

The method can include wherein said one or more non-reducing disaccharides are selected from the group consisting of: sucrose, trehalose, and isomalt.

The method can include wherein said monosaccharide derivatives are selected from the group consisting of: methylglucoside, and 2-Deoxy-d-glucose.

The method can include wherein said sugar alcohols are selected from the group consisting of: glycerol, sorbitol, mannitol, and erythritol.

The method can include wherein the maximum storage temperature is 40° C., and the glass transition temperature is greater than or equal to 41° C.

The method can include wherein the ionizing radiation dose comprises electron beam irradiation, gamma irradiation, X-ray irradiation, or ultraviolet light.

The method can include wherein the vaccine is thermostable if activity decreases less than 0.5 logs after: (i) 1 year of storage at room temperature, (ii) 3 months of storage at 37° C., and (iii) after 1 hour at 70° C.

The method can include wherein the live virions comprise: coronavirae, influenza, rabies, measles, rubella, yellow fever, smallpox, respiratory syncytial, herpes, or aids.

The method can include wherein the cellular microorganisms comprise bacteria, fungi, vibrio, or yeast.

The method can include wherein the cellular microorganisms comprise anthrax, Listeria shigella, salmonella, E. coli, Yersinia pestis, or cholera.

The method can be enhanced to further comprise isolating nucleic acid aptamers specific to the inactivated or killed vaccine.

The enhanced method can include wherein said isolating comprises contacting particles of the inactivated or killed vaccine with nucleic acid aptamers and filtering retentate, wherein the retentate comprises complexes of said particles and nucleic acid aptamers.

The enhanced method can further include selecting a plurality of therapeutic aptamer candidates from the retentate.

The enhanced method can further include sequencing the therapeutic aptamer candidates.

The enhanced method can further include synthesizing and purifying at least one of the therapeutic aptamer candidates to form a therapeutic composition.

The enhanced method can further include confirming specific binding of the therapeutic composition to the virions or bacterium of the microorganism preparation, wherein said binding is performed in human serum.

The enhanced method can further include identifying non-neutralizing aptamers of the plurality of therapeutic aptamer candidates.

The enhanced method can further include combining the non-neutralizing aptamers with the inactivated or killed vaccine to form an enhanced vaccine preparation.

In another embodiment, a method of producing a dry thermostable inactivated vaccines and biopharmaceuticals from a suspensions of pathogenic microorganisms (virions and cellular microorganisms), comprises: (i) stabilizing a suspension of pathogenic virions or cellular microorganisms at ambient temperatures by immobilizing the microorganisms in a protective glassy matrix with glass transition temperature greater than a maximum ambient storage temperature comprised of two or more protective molecules which could be carbohydrates, amino acids, silica, their derivatives or/and polymers, and (ii) subsequently exposing the stabilized microorganisms to an ionizing radiation dose between 24 kGy and 48 kGy at ambient temperatures to decrease the survival of the microorganisms more than a million times and to produce an inactivated or killed but potent products. Additionally, this method may optionally include where the stabilization comprises: suspending the microorganisms in a preservation solution containing the protective molecules; primary drying by vaporization or evaporation (desorption) at ambient temperatures from −20° C. to +40° C.; and secondary drying at elevated temperatures above 40° C. with subsequent cooling to an ambient storage temperature below 40° C.

In yet another embodiment, a method of primary drying by vaporization may include where the vaporization drying is performed under vacuum from a partially frozen slush state in the presence of ice crystals in the suspension to make the process better scalable, reproducible, and executable using a conventional lyophilizer.

The disclosure, in its various aspects and embodiments, suggests (i) thermo-stabilization of wild type infectious microorganisms, including without limitation SARS-COV-2 virus, by first immobilizing the microorganisms in a protective carbohydrate or silica-glass, then (ii) subsequent inactivation with a relatively high dose of irradiation, such as electron beam (EB) inactivation, or inactivation using other sources of irradiation, including without limitation gamma, x-ray, UV light, and the like. Such will produce inactivated and potent vaccines.

EB inactivation has been found to inactivate the vaccine composition through virus nucleic acid damage without affecting virus surface structures immobilized in the glass environment, thus preserving integrity of epitopes and providing an accurate template for vaccine and aptamer therapy production.

Also suggested is the selection of high affinity “neutralizing” aptamers (short segments of DNA, RNA or peptide that binds to a specific target) at the surface of EB-inactivated microorganisms (e.g. SARS-COV-2 virus or other microorganism templates). These neutralizing aptamers can be used as therapeutics to treat disease. Aptamers offer several advantages over other types of therapeutics (such as antibodies) because of their relatively small physical size, flexible structure, fast chemical production, versatile chemical modification, high stability, and lack of immunogenicity.

The methods described and claimed herein are differentiated at least because the irradiation is applied to microbes that have already been thermostabilized through a dry thermo-stabilization process (e.g., preservation by vaporization (PBV)), eliminating damaging effects of free radicals that are formed during irradiation of a product in a liquid state. Moreover, it is further suggested that a neutralizing aptamer therapeutic against pathogenic microbes could be developed using ionizing radiation-inactivated dry preserved microbes. Selecting aptamers against a whole microbe to develop a therapeutic is unconventional and offers the advantage of selecting several high affinity ligand molecules (polyclonal aptamers) that bind to different surface targets of the microbe (e.g., virion) in their native conformation and physiological environment, without requirement for target protein isolation and purification. Although conventional aptamer selection is conducted in model media, such as phosphate buffered saline (PBS), it is herein proposed to perform the selection in human blood serum (HBS) to ensure retention of aptamers that are more resistant to DNAse attack in blood.

Current full vaccine development strategies require many months, or even years to complete. Such is an unsustainable timeline when faced with emerging diseases like COVID-19. Proposed herein, in one example, is an accelerated approach for production of a safe, thermostable, and potent vaccine against COVID-19 by electron beam inactivation of wild type SARS-COV-2 virus immobilized in a carbohydrate glass through use of preservation by vaporization (PBV) drying technology.

Wild type virus inactivation has been successfully used for vaccine production. Well known examples are chemically (e.g., formaldehyde) inactivated Salk polio, eastern equine encephalitis vaccine, and tick-born encephalitis vaccines. However, there are cases where chemical activation of certain vaccines (e.g., measles and respiratory syncytial virus (RSV)) leads to an enhanced or atypical course of the disease after subsequent vaccination. Several chemically inactivated vaccine candidates against coronaviruses were found to cause Th2-type immunopathology and induce antibody-dependent enhancement (ADE) of infectivity and eosinophilia. The immunopathological effects of ADE are characterized by enhancement of viral entry and induction of a severe inflammatory response. Recently, studies on patients with severe COVID-19 have found an increased IgG response, which is an indication of possible ADE of SARS-COV-2 infection. These observations could be used to question one's approach for development of a vaccine against COVID-19 by inactivating wild type virus. Mechanisms of ADE are not clearly established. For inactivated RSV vaccine candidates, it was found that atypical course of the disease after subsequent vaccination was associated with formaldehyde disrupting key protein epitopes during the inactivation process. Alternatively, gamma-ray and electron beam irradiation have the ability to inactivate pathogens by damaging nucleic acids rather than proteins, leaving key epitopes undamaged, suggesting these technologies may be suitable methods for inactivation of virus for vaccine production. Also, it was previously demonstrated that electron beam inactivation of RSV generates a potent vaccine with no ADE symptoms in mice, which indicates that the ADE induction could have been because the vaccines had been produced by chemical inactivation, which damaged viral antigenic determinants.

The methods disclosed and claimed herein, including EB inactivation applied to SARS-COV-2 virus or other microorganisms, which are first stabilized into a carbohydrate-glass according to a thermo-stabilization process (e.g., PBV), can generate potent and thermostable vaccines. Based on results of preliminary data, it is expected that after inactivation there will be no or minimal changes in the viral antigenicity and viral surface. Hence, the inactivated whole virus, according to the methods herein, could also be used for development of antibody and/or aptamer therapeutics. Use of the intact surface of EB inactivated microbes is advantageous, for example, because using live SARS-COV-2 for this purpose would raise biosafety concerns and cost. Another advantage makes use of the observation that aptamers are substantially less immunogenic than antibodies, which as stated above might induce ADE.

For aptamer development, it is suggested to use a modified systematic evolution of ligands by exponential enrichment (SELEX) methodology to develop high affinity neutralizing polyclonal aptamers for inactivated SARS-COV-2 and evaluate their ability to neutralize live SARS-COV-2 virus infectivity in vitro and in vivo. The aptamers may then be researched as therapeutic candidates to treat and, potentially prevent, SARS-COV-2 infection. This process requires about a month to select high affinity neutralizing aptamers, which is much faster than the conventional required time (˜1 year) needed for development of good neutralizing antibodies.

### Example 1: Potent Thermostable Inactivated Rabies Live Attenuated Vaccine ERA-333 with Substantially Improved Safety

PBV and EB inactivation was successfully applied to formulate a potent thermostable inactivated animal rabies live attenuated vaccine ERA-333 with substantially improved safety for use in human applications. Results of the challenge studies in mice are shown in Table 1.

TABLE 1
Commercial Rabies Vaccine with EB-inactivated PBV Vaccine

| | PBV Live Attenuated | | | | | Commercial | PBV & EB Radiated | | |
|---|---|---|---|---|---|---|---|---|---|
| | Vaccine | | | ERAg333ª | Placebo | Vaccine | Inactivated Vaccine | | |
| Group | 1 | 2 | 3 | 4 | 5 | 6 | 7 | 8 | 9 |
| Dilution | 10⁻¹ | 10⁻² | 10⁻³ | 10⁻² | None | None | None | 10⁻¹ | 10⁻² |
| Titer/Load | 6.8^{b} | 5.7 | 4.4 | 7.9 | 300^{b} | 620 | 350 | 34 | 2.3 |
| GMT day 14^{c} | 0.26 | 0.11 | 0.20 | 0.37 | <0.05 | 0.23 | 0.07 | <0.05 | <0.05 |
| GMT day 30^{c} | 1.60 | 0.96 | 1.70 | 0.84 | <0.05 | 0.58 | 0.27 | 0.13 | <0.05 |
| Survival^{d} | 100% | 100% | 100% | 100% | 22% | 100% | 100% | 100% | 80% |

^{a}Parent strain for both live attenuated and inactivated vaccines; generated by reverse genetics.
^{b}0.1 ml dose.
^{c}Geometric mean titer (GMT) (IU/mL) of rabies virus neutralizing antibodies.
^{d}Group size = 10 except placebo n = 9.

High potency of EB inactivated vaccines observed in these studies reflects very small decreases in the viral antigenicity after EB inactivation. In other recent studies, EB inactivation was applied to produce highly antigenic thermostable inactivated polio and influenza vaccine candidates.

### Example 2: Potent Thermostable Inactivated Live Attenuated Influenza H3N2 Virus (LAIV) with Substantially Improved Safety

Similar results to those of Example 1, but with respect to live, attenuated influenza H3N2 virus (LAIV), were observed. In this study, using PBV, thermostable live attenuated influenza vaccine LAIV (H3N2) was produced. PBV LAIV stability after a year of storage at room temperature (RT) and at 37° C. is shown in Table 2.

TABLE 2
Activity PBV -Preserved LAIV After Reconstitution

| Storage | | | Average | Average | Average |
|---|---|---|---|---|---|
| (months) | Storage Temp | Log loss | (TCID₅₀/mL) | (TCID₅₀/mg) | (TCID₅₀/mg) |
| Initial Yield | Frozen Control | N/A | 8.95E+07 | 1.86E+05 | 6.79E+04 |
| | After PBV | −0.41 | 3.44E+07 | 7.17E+04 | 0.99E+04 |
| 1 | Frozen Control | N/A | 3.04E+07 | 6.33E+04 | 3.69E+04 |
| | RT | −0.61 | 1.48E+07 | 1.55E+04 | 0.20E+04 |
| | 37° C. | −0.56 | 1.67E+07 | 1.74E+04 | 0.60E+04 |
| 12 | Frozen Control | N/A | 2.51E+07 | 5.23E+04 | 0.00E+04 |
| | RT | −0.65 | 5.67E+06 | 1.18E+04 | 0.19E+04 |
| | 37° C. | −0.85 | 3.58E+06 | 7.46E+03 | 0.12E+04 |

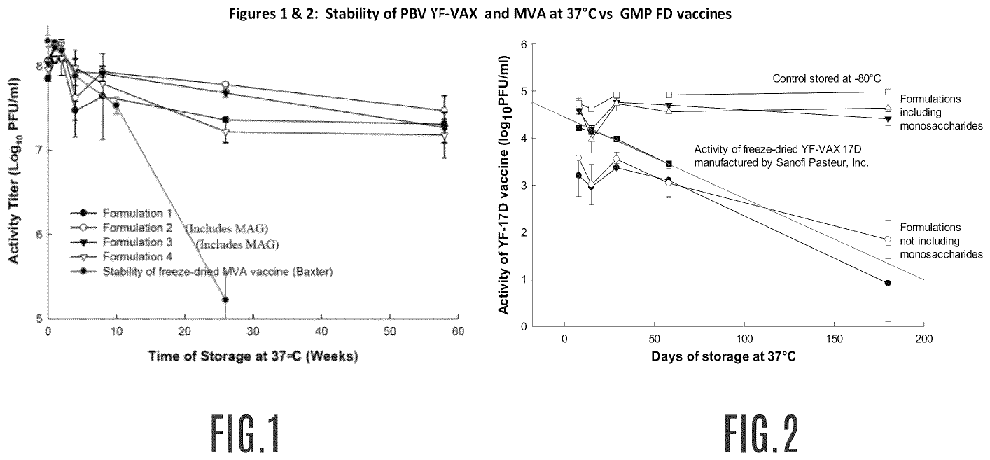

Furthermore, it was demonstrated that EB could be used to effectively inactivate LAIV with no significant loss of the antigenicity measured by Hemagglutination Inhibition (HI) Assay (see Table 3). For example, FIGS. 1 and 2 support the finding that preservation by vaporization (PBV) is preferable over freeze drying (FD) for stabilization of vaccines, such as smallpox (Modified Vaccinia Ankara (MVA)) and yellow fever vaccines. In these studies, in order to ensure direct unbiased comparison of PBV and FD, FD YF-VAX-17D (Sanofi) and MVA (Baxter) vaccines produced under GMP for human application were reconstituted, and subsequently dried using PBV with no activity loss after drying. PBV vaccines remained stable at 37° C. for at least 6 months, while FD vaccine activity decreased 1000 times.

TABLE 3
Effect of EB Dose on Inactivated PBV Live Attenuated
Influenza Virus Activity and Antigenicity (HI)

| | | Titer | HI Titer |
|---|---|---|---|
| EB Dose | Log loss | (TCID₅₀/mL) | (geometric mean) |
| Frozen Control | N/A | 6.31E+07 | 320 |
| After PBV (0 kGy) | −0.35 | 2.80 ± 0.36E+07 | 320 |
| 6 kGy | −3.20 | 3.97 ± 1.37E+04 | 320 |
| 12 kGy | −4.51 | 1.93 ± 0.44E+03 | 320 |
| 18 kGy | −5.14 | 4.59 ± 1.5E+02 | 320 |
| 24 kGy | −6.52 | 1.90E+01 | 320 |
| 48 kGy | N/A | N/A | 285 |

### Example 3. PBV Ebola, Live Attenuated Rabies, and Other Live Attenuated Vaccines (LAVs)

PBV technology was used to achieve a stable Ebola virus vaccine candidate that retained 100% potency after one year storage at 37° C., a live-attenuated Rabies vaccine that remained stable for at least 23 months at RT without losing its protective efficacy, and many other LAVs. The PBV process can be executed using any conventional freeze-drying equipment by reprograming the computer that controls the vacuum drying protocol.

### Example 4. Production of Thermostable Microorganisms Using PBV

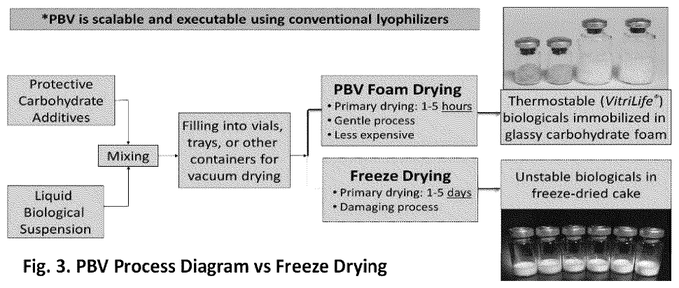

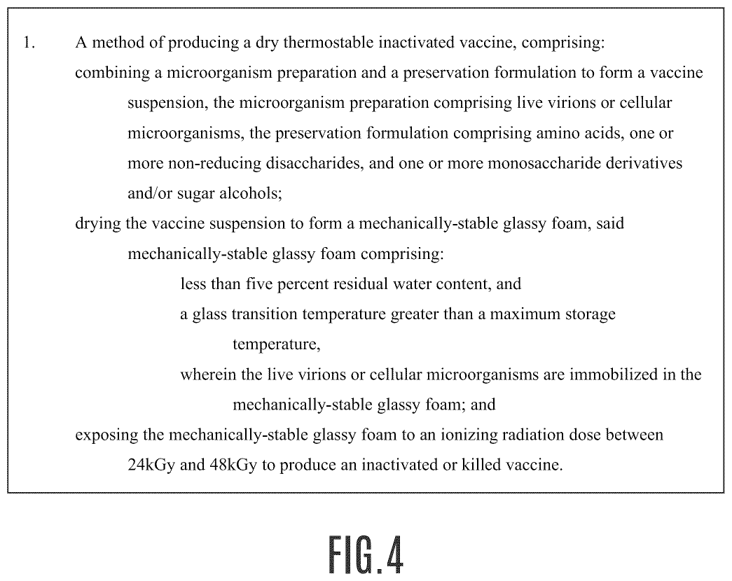

The PBV process starts by mixing a microorganism suspension with a preservation solution (PS) comprising non-reducing carbohydrates protecting vaccines from desiccation stress during PBV. As illustrated in FIG. 3, dehydration is achieved by boiling under vacuum from a partially frozen state (i.e. slush) at near subzero temperatures (several degrees below 0° C.), followed by secondary drying at elevated temperatures above 40° C.

Primary PBV drying typically takes several hours during which the preservation mixture (PM) is transformed into mechanically stable glassy foam. Secondary drying by desorption of remaining water from the foam is required to increase the glass transition temperature (T_{g}) inside the foam above 40° C., immobilizing the viruses (or other microbes) in a foamed sugar matrix for stability at ambient temperatures (AT). A PBV-preserved microbe (virus, etc.) is thermostable if its survival decreases less than 0.5 logs after: (i) 1 year of storage at room temperature, (ii) 3 months of storage at 40° C. or 37° C., and (iii) after 1 hour at 70° C.

The PBV process is a complex multi-factorial process. Various controllable and uncontrollable factors reflecting both the preservation treatment and the physiological characteristics of the virus or other microbe will affect survival and stability of PBV formulations. Before drying, the vaccine will be mixed with a preservation solution to form preservation mixtures. Factors characterizing the preservation process for vaccine stabilization include composition and concentration of preservation solutions and parameters of the PBV drying protocol. A challenge is to achieve the optimized thermostable formulations. However, optimized thermostable formulations could be achieved using following working hypotheses: First, effective long-term preservation of microorganisms (i.e. virions, bacterium, etc.) requires immobilization of the microorganisms in a dry glass carbohydrate matrix (e.g. mechanically stable foam). The glass transition temperature (T_{g}) of the matrix should be higher than the maximum temperature at which the preserved microorganisms will be stored. Second, drying may not be a direct damaging factor to a microorganism structure and function. Some of the dehydration-induced damage to unprotected microorganisms is associated with hydration forces that rise between biological membranes and macromolecules when the distance between them becomes very small. This dehydration-induced damage can be diminished by using preservation solutions (PS) comprising protective carbohydrates that preferentially adsorb at the surface of biological membranes and macromolecules. The protective carbohydrates will eliminate hydration forces during drying and create glassy shells immobilizing the biological membranes and macromolecules. The list of protective carbohydrates includes without limitation non-reducing disaccharides, monosaccharide derivatives, and sugar alcohols. Also, PSs should be formulated to increase stability of the amorphous state after drying. It is preferred that the concertation of disaccharides in PSs is at least twice (more preferably, 3 times) higher than that of monosaccharide derivatives, or sugar alcohols. In this regard, the preservation formulation should comprise one part by weight monosaccharide derivatives and/or sugar alcohols and at least two parts by weight non-reducing disaccharides.

Based on this, thermostable live attenuated anthrax, Listeria shigella, salmonella, cholera, influenza, rabies, measles, rubella, yellow fever, small pox, RSV, and many other vaccines and microbiome bacteria were prepared using preservation solutions comprising mixtures of amino acids, non-reducing disaccharides, monosaccharide derivatives, and sugar alcohols. To protect membranes and envelopes of microorganisms during glassification by drying we used molecules that preferentially adsorb at the surface of microorganisms including some polymeric protectants and silica. To protect intracellular macromolecules, we loaded microorganisms with protective non-reducing derivative of monosaccharides and sugar alcohols before drying. Cellular microorganisms are loaded by adding the protective carbohydrates in the microorganism growth media, and microorganisms were grown in the loaded media.

### Example 5. Inactivation of PBV Preserved Viruses

EB inactivation was performed without opening the sealed serum vials containing PBV preserved live polio and influenza viruses. It was discovered that to decrease viral activity above 1 million times without loss of antigenicity, EB inactivation doses should be above 24 kGy, but below 48 kGy where a small decrease in antigenicity was detected (Table 3). Because of that, to inactivate wild infectious microorganisms, an EB inactivation dose above 24 kGY but below 48 kGy is preferred.

### Example 6. Development High Affinity Aptamers Specific to the EB Inactivated Vaccine Candidate Against COVID-19

Nucleic acid aptamers are short, single-stranded (ss) DNA or RNA molecules that are selected for binding to a specific target. Nucleic acid aptamers are often termed “chemical antibodies.” Like antibodies, due to complex ligand-receptor recognition collective interactions, aptamers bind to antigens and can also interfere with normal physiological function of certain target antigens and act similarly to neutralizing bodies. For example, in the case of SARS-COV-2, aptamers may shield virus surface spike structures and thus prevent recognition of the cellular ACE2 receptors.

“Polyclonal” aptamers capable of high affinity and specificity binding to the EB inactivated thermostable SARS-COV-2 target, which will serve as a template for the aptamer selection, could be selected using several techniques.

Original SELEX methodology for generating aptamers for protein antigens was previously described. It has been demonstrated that aptamers with high affinity and specificity could be selected not only for protein antigens but also for viruses, bacteria and other cells. For example, aptamers were identified for SARS COV and for the Hepatitis C virus. To isolate DNA aptamers specific to the inactivated SARS-COV-2 (the vaccine) one with skill in the art can adapt a method of fast aptamer isolation recently applied by V. Gilman in selection of aptamers for pathogens found in platelet concentrates. The EB-inactivated SARS-COV-2 will serve as the template for aptamer selection of target vaccine particles (TVP). The process of aptamer isolation includes the following main steps: (i) aptamer library-TVP contact, (ii) isolation and identification of best binding candidates, and (iii) verification of specific binding.

### Aptamer Library-TVP Contact.

The selection can be conducted in filtered human blood serum (HBS) to ensure retention of aptamers specific to the target and resistant to DNAse attack in blood. HBS is filtered using 100 kDa cut-off Amicon Ultra-15 centrifuge filter, equipped with ultralow binding Ultracel membrane (Millipore, FC9 100 08). Thus, the resultant filtrate may contain major parts of HBS components and all blood DNAses. The starting library of DNA oligonucleotides (˜1015 random sequences) may be purchased from Integrated DNA Technologies (IDT, Iowa, USA). The library can contain custom synthesized 80-mer ssDNA oligonucleotides containing two flanking primer-binding regions and a random middle segment (N38). The library oligonucleotide sequence motif can have an average molecular weight of 24.5 kDa. The ssDNA oligonucleotide library is dissolved in 2.0 mL of Phosphate Buffered Saline (PBS) pH 7.4. The library is mixed with 10 mL of HBS containing approximately 109 TVP. The mixture is gently aspirated and incubated 30 min at 37° C. Equilibration time (30 min) was selected because average half time of aptamers in serum is around 60 minutes. After incubation, the mixture is dispensed into Sartorius 300 kDa cut-off, Vivaspin 500, 300, 000 MWCO units (estimated pore size 10.0 nm) and filtered-centrifuged. The retentate collected in the filter beds (˜5.0 μL) is successively washed ×11 with individual 0.5 mL volumes of PBS pH 7.4 in each filter. This results in a calculated 1:1022 dilution of the initial ssDNA library containing approximately 1015 individual sequences. After the final PBS wash, the retentate will contain TVP-ssDNA complexes that survived the high dilution wash. The retentates obtained in all filters are combined. It should be noted that, in the above procedure, HBS will be originally filtered using a 100 kDa cut-off membrane; however, the post TVP and ssDNA contact treatment will be conducted using a 300 kDa cut-off membrane. Therefore, if by-product complexes of HBS components with ssDNA oligonucleotides exceeding 100 kDa cut-off are formed, they will be removed during the target-aptamer post contact treatment.

### Isolation and Identification Best Binding Candidates.

The combined retentate containing TVP-ssDNA complexes are transferred into a new 300 kDa filter, added with 0.4.0 of sodium thiocyanate (2.0 M), gently aspirated, and incubated at RT for 30 min to elute surface bound aptamer candidates. The filter is centrifuged, and the filtrate containing the aptamer candidates is collected. The filtrate then is desalted using a 5 kDa Amicon centrifugal filter with 5 PBS washings. The retentate will contain aptamers.

The sequences of the collected aptamer candidates will be identified by molecular cloning using pCR2.1 TOPO TA cloning method (Invitrogen). Specifically, the ssDNA aptamer pool can be amplified by PCR (2 min at 95° C.; 30 cycles of 95° C. for 30 sec, 52.8° C. for 30 sec, 72° C. for 30 sec; final extension at 72° C. for 5 min) using the adapter region primers (Const fwd and Const rev). The PCR amplicon pool can be purified with the Gel extraction kit (Qiagen, Maryland) and cloned into pCR2.1 TOPO vector using TOPO TA cloning kit (Thermo Fisher Scientific, California). The ligated vectors can be transformed into One Shot® Top10 chemically competent E. coli cells (Invitrogen) and transformed bacteria can be plated on Luria Bertani (LB) agar plates with kanamycin (50 μg/ml) and incubated at 37° C. for 18 hr. Individual colonies of the transformed cells can be propagated in 10 ml LB broth with kanamycin (50 μg/ml) for 16 hr at 37° C. The cells can be harvested by centrifugation and plasmid DNA can be extracted using the QIAprep® Spin plasmid Miniprep Kit. The colonies can be screened by PCR using M13 forward and M13 reverse primers. The size of amplified products can be visualized using 2% agarose gel electrophoresis. As the empty vector contains 201 bp, the transformants with an amplicon band size of 281 bp are selected for sequencing.

The DNA sequences of selected colonies can be identified by Sanger sequencing method as a service, at IDT. Once the sequences of the selected aptamer candidates are determined, ranking of these sequences can be conducted using expected changes in Gibbs free energy (ΔG) and melting temperature (Tₘ) calculated by predicted folding at 37° C. using settings representing average blood ion strength and composition (MFold (unafold.rna.albany.edu)). Sequences of several aptamers, showing predicted combinations of lowest ΔG values and highest values of Tₘ, can be selected for chemical synthesis and purification (as a service, IDT). Some 2.5-mg quantities of the purified individual candidates may be generated.

### Verification of Specific Binding.

Specificity of the identified aptamers to SARS-COV-2 is confirmed using direct aptamer binding to TVP in human blood. To this end, citrated human blood is spiked with different titers of TVP. To these suspensions, a defined amount of the aptamer candidate solutions in PBS pH 7.4 are mixed in. Upon incubation at 37° C., the mixtures are centrifuged at a high speed sufficient for sedimentation of TVP. The concentration of the unbound aptamer candidate is determined in the supernatant using qRT-PCR. Specific binding should result in TVP dose specific response. A goal is to select aptamers that decrease the viral infectivity at least 100 times.

Separation of Aptamers that do not Neutralize Viral Ability to Infect Cells.

Not all aptamers from the best binding group selected will inactivate viral ability to infect cells (not all are neutralizing). These non-neutralizing aptamers can be identified and separated from the mixture to produce better aptamer therapeutics. The separated non-neutralizing aptamers can be mixed with inactivated viruses to produce an enhanced vaccine that will induce neutralization antibodies but ill not produce antibodies against epitopes covered with non-neutralizing aptamers.

Therefore, in accordance with the disclosure, methods for producing dry thermostable inactivated vaccines with improved inactivation/killing without compromising antigenicity are disclosed. The methods provide for microbes to be irradiated with an ionizing radiation dose above 24 kGy without compromising the integrity of useful epitopes. To achieve enhanced inactivation, microorganisms (virions, bacterium, fungi, etc.) are first immobilized in a dry glassy matrix, then subsequently irradiated at elevated radiation dose while immobilized in the glass. Aptamers can be isolated from the inactivated microbes, such as inactivated SARS-COV-2, and modified or developed as therapeutics for neutralizing infectious disease. Additionally, non-neutralizing aptamers can be combined to produce an enhanced vaccine.

Finally, it should be recognized that the preservation mixtures, and resulting compositions for vaccines and therapeutics, may comprise a number of alternatives of protective salts, amino acids, and carbohydrates which are not described in the examples. One having skill in the art, along with the conventional knowledge in the art, and the description herein, will be adequately enabled to make and use the claimed invention without undue experimentation. As such, the above-described examples are intended to be non-limiting of the spirit and scope of the claimed invention.

What is claimed is:

1. A method of producing a dry thermostable inactivated vaccine, comprising:
combining a microorganism preparation and a preservation formulation to form a vaccine suspension, the microorganism preparation comprising live virions or cellular microorganisms, the preservation formulation comprising amino acids, one or more non-reducing disaccharides, and one or more monosaccharide derivatives and/or sugar alcohols;
drying the vaccine suspension to form a mechanically-stable glassy foam, said mechanically-stable glassy foam comprising:
less than five percent residual water content, and
a glass transition temperature greater than a maximum storage temperature,
wherein the live virions or cellular microorganisms are immobilized in the mechanically-stable glassy foam; and
exposing the mechanically-stable glassy foam to an ionizing radiation dose between 24 kGy and 48 kGy to produce an inactivated or killed vaccine.

2. The method of claim 1, wherein the preservation formulation comprises one part by weight monosaccharide derivatives and/or sugar alcohols and at least two parts by weight non-reducing disaccharides.

3. The method of claim 2, wherein said one or more non-reducing disaccharides are selected from the group consisting of: sucrose, trehalose, and isomalt.

4. The method of claim 2, wherein said monosaccharide derivatives are selected from the group consisting of: methylglucoside, and 2-Deoxy-d-glucose.

5. The method of claim 2, wherein said sugar alcohols are selected from the group consisting of: glycerol, sorbitol, mannitol, and erythritol.

6. The method of claim 1, wherein the maximum storage temperature is 40° C., and the glass transition temperature is greater than or equal to 41° C.

7. The method of claim 1, wherein the ionizing radiation dose comprises electron beam irradiation, gamma irradiation, X-ray irradiation, or ultraviolet light.

8. The method of claim 1, wherein the vaccine is thermostable if activity decreases less than 0.5 logs after: (i) 1 year of storage at room temperature, (ii) 3 months of storage at 37° C., and (iii) after 1 hour at 70° C.

9. The method of claim 1, wherein the live virions comprise: coronavirae, influenza, rabies, measles, rubella, yellow fever, smallpox, respiratory syncytial, herpes, or aids.

10. The method of claim 1, wherein the cellular microorganisms comprise: bacteria, fungi, vibrio, or yeast.

11. The method of claim 10, wherein the cellular microorganisms comprise: anthrax, Listeria shigella, salmonella, E. coli, Yersinia pestis, or cholera.

12. The method of claim 1, further comprising: isolating nucleic acid aptamers specific to the inactivated or killed vaccine.

13. The method of claim 11, wherein said isolating comprises contacting particles of the inactivated or killed vaccine with nucleic acid aptamers and filtering retentate, wherein the retentate comprises complexes of said particles and nucleic acid aptamers.

14. The method of claim 12, further comprising selecting a plurality of therapeutic aptamer candidates from the retentate.

15. The method of claim 13, further comprising sequencing the therapeutic aptamer candidates.

16. The method of claim 14, further comprising synthesizing and purifying at least one of the therapeutic aptamer candidates to form a therapeutic composition.

17. The method of claim 15, further comprising confirming specific binding of the therapeutic composition to the virions or bacterium of the microorganism preparation, wherein said binding is performed in human serum.

18. The method of claim 16, further comprising identifying non-neutralizing aptamers of the plurality of therapeutic aptamer candidates.

19. The method of claim 17, further comprising combining the non-neutralizing aptamers with the inactivated or killed vaccine to form an enhanced vaccine preparation.

20. A method of producing a dry thermostable inactivated vaccines and biopharmaceuticals from a suspension of pathogenic microorganisms, comprising:
stabilizing a suspension of the pathogenic microorganisms at ambient temperatures by immobilizing the microorganisms in a protective glassy matrix with glass transition temperature greater than a maximum ambient storage temperature comprised of two or more protective molecules which could be carbohydrates, amino acids, silica, their derivatives or/and polymers, and
subsequently exposing the stabilized microorganisms to an ionizing radiation dose between 24 kGy and 48 kGy at ambient temperatures to decrease the survival of the microorganisms more than a million times and to produce an inactivated or killed but potent products.
